(19) United States
(12) Patent Application Publication
(10) Pub. No.: US 2007/0253922 A1
(43) Pub. Date: Nov. 1, 2007

(54) ANHYDROUS ANTIPERSPIRANT/DEODORANT STICK COMPOSITIONS

(75) Inventor: Akitsu Taira, Sumida-ku (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Kao Corporation, Chuo-ku (JP)

(21) Appl. No.: 11/738,037

(22) Filed: Apr. 20, 2007

(30) Foreign Application Priority Data
Apr. 28, 2006 (JP) 2006-126334

(51) Int. Cl. A61K 8/28; A61K 8/26

(52) U.S. Cl. 424/66; 424/68

(57) ABSTRACT

An anhydrous antiperspirant/deodorant stick composition containing the following ingredients (A), (B), (C) and (D): (A) from 15 to 30 wt % of a higher alcohol having from 12 to carbon atoms, (B) a polypropylene glycol having an average molecular weight from 400 to 2,000, (C) a volatile cosmetic oil, and (D) an antiperspirant agent or deodorant agent.

### FIELD OF THE INVENTION

This invention relates to stick-shaped, anhydrous antiperspirant/deodorant compositions.

### BACKGROUND OF THE INVENTION

Stick-type antiperspirant/deodorant compositions are widely used, and those making a combined use of an aluminum and/or zirconium salt, a higher alcohol, a volatile silicone and the like are in common use. However, these antiperspirant/deodorant sticks may not be fully satisfactory in its feeling upon use, and attempts have been made to improve their feeling upon use together with their antiperspirant/deodorant effects.

For example, in U.S. Pat. No., 4,725,432, a long-chain fatty alcohol having 20 or more carbon atoms was used to provide a solid stick with improved hardness. In U.S. Pat. No. 5,932,199, an attempt was made to improve the feeling upon use by using a perfume carrier material and adding a cream which contains one or more humectants such as glycerin.

Nonetheless, there has been a desire for antiperspirant/deodorant stick compositions having an improved feeling upon use.

### SUMMARY OF THE INVENTION

The present invention provides an anhydrous antiperspirant/deodorant stick composition containing the following ingredients (A), (B), (C) and (D):
(A) from 15 to 30 wt % of a higher alcohol having from 12 to 26 carbon atoms,
(B) a polypropylene glycol having an average molecular weight of from 400 to 2,000,
(C) a volatile cosmetic oil, and
(D) an antiperspirant agent or deodorant agent.

### DETAILED DESCRIPTION OF THE INVENTION

The present invention relates to an antiperspirant/deodorant stick compositions excellent in feeling upon use.

The present inventor has found that an anhydrous antiperspirant/deodorant stick composition, which provides an adequate dry feel, gives a smooth application feel without stickiness and is reduced in the production of a white residue, can be obtained by using a polypropylene glycol of a particular molecular weight in combination with a higher alcohol, a volatile cosmetic oil and an antiperspirant agent or deodorant agent.

The anhydrous antiperspirant/deodorant stick composition according to the present invention is excellent in feeling upon use, specifically provides an adequate dry feel, is free of stickiness, gives a smooth application feel, and does not produce much white residue.

The higher alcohol for use as ingredient (A) in the present invention is one having from 12 to 26 carbon atoms, preferably from 16 to 24 carbon atoms, more preferably from 18 to 22 carbon atoms. Within this range, a good feel to the touch can be obtained. Specific higher alcohols include cetyl alcohol, stearyl alcohol, arachidyl alcohol, behenyl alcohol, and the like.

As the higher alcohol (A), such higher alcohols can be used either singly or in combination. The higher alcohol (A) is contained at from 15 to 30 wt %, preferably from 17 to 25 wt % based on the whole composition. Within this range, a good feel to the touch can be obtained along with adequate formability.

The polypropylene glycol for use as ingredient (B) in the present invention is one having an average molecular weight of from 400 to 2,000 (degree of polymerization: 7 to 34), preferably from 400 to 1,200 (degree of polymerization: 7 to 20), more preferably from 400 to 1,000 (degree of polymerization: 7 to 18). Within this range, a good feel to the touch can be obtained.

As such a polypropylene glycol, a commercial product such as “NEWPOLPP-400”, “NEWPOLPP-1000” or “NEWPOLPP-2000” (product of Sanyo Chemical Industries, Ltd.) can be used.

As the polypropylene glycol (B), one or more of such polypropylene glycols can be used. The polypropylene glycol (B) maybe contained preferably from 3 to 15 wt %, more preferably from 4 to 10 wt % based on the whole composition, because a good feel to the touch can be obtained.

As the volatile cosmetic oil for use as ingredient (C) in the present invention, paraffinic hydrocarbons, isoparaffinic hydrocarbons, volatile silicones and the like can be used, with volatile silicones being preferred. The term “volatile” as used herein means a property that a vapor pressure is measurable at 25° C. The volatile cosmetic oil may generally have a vapor pressure at 25° C. of from 0.01 to 6 mmHg, preferably from 0.02 to 1.5 mmHg, and a boiling point at 1 atm of lower than 250° C.

These preferred volatile silicones include cyclic silicones, straight-chain silicones, branched-chain silicones, and the like. More preferred volatile silicones are cyclic silicones represented by the following formula: 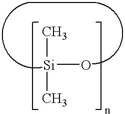
wherein n stands for a number of from 3 to 7, preferably from 4 to 6, more preferably 5.

Such volatile cyclic silicones generally have viscosities of 10 mm²/s or lower, respectively. As volatile silicones, it is possible to use commercial products such as “DOW CORNING 244”, “DOW CORNING 245”, “DOW CORNING 344” and “DOW CORNING 345” (all, trade names, products of Dow Corning Corporation), “SH245” (trade name, product of Dow Corning Toray Co., Ltd.), “SILICONE FLUIDS SF-1202” and “SILICONE FLUIDS SF-1173” (both, trade names, products of General Electric Company), “KF994” and “KF995” (both, trade names, products of Shin-Etsu Chemical Co., Ltd.). It is also possible to use the volatile silicones described in Todd et al., “Volatile Silicone Fluids for Cosmetics”, Cosmetics and Toiletries, 91, 27-32 (1976).

As the volatile cosmetic oil (C), one or more of such volatile cosmetic oils can be used. The volatile cosmetic oil (C) maybe contained preferably at from 5to 70 wt %, more preferably at from 10 to 60 wt % based on the whole composition, because a good feel to the touch can be obtained.

In ingredient (D) for use in the present invention, the antiperspirant agent may preferably be in a solid form. It is preferred to disperse the antiperspirant agent as fine particles in the composition, and the average particle size of the fine particles may be preferably from about 0.1 to 100 μm, with a range of from 0.1 to 20 μm being more preferred and a range of from 0.1 to 10 μm being even more preferred.

Examples of antiperspirant agents usable in the present invention include astringent metal salts such as inorganic salts and organic salts of aluminum, zirconium, zinc and the like. Among these, preferred are aluminum salts and/or zirconium salts such as aluminum halides, aluminum hydroxyhalides, zirconium halides, zirconium oxyhalides, and zirconium hydroxyhalides.

As the aluminum salts, preferred are those represented by the following formula:
Al₂(OH)ₐCl_{b}.xH₂O
wherein a stands for a number of from 2 to 5, the total of a and b is 6, and x stands for a number of 1 to 6. More preferred are aluminum chlorohydrates (aluminum chlorohydroxides) of the above formula in which a is 4 and b is 2.

Such aluminum salts can be produced, for example, by the process disclosed in U.S. Pat. No. 3,887,692, U.S. Pat. No. 3,904,741 or the like. Further, the aluminum salt mixtures disclosed in G.B. Patent No. 2,048,299 can also be used.

As the zirconium salts, preferred are those represented by the following formula:
ZrO(OH)₂₋ₐClₐ.xH₂O
wherein a stands for a number of from 1.5 to 1.9, and x stands for a number of 1 to 7.

More preferred are complexes (ZAG complexes) of aluminum zirconium chlorohydrates, which contain aluminum and zirconium, and glycine.

Preferred antiperspirant agents include aluminum chlorohydrate, aluminum dichlorohydrate, aluminum sesquichlorohydrate, aluminum chlorohydrate/propylene glycol complex, aluminum dichlorohydrate/propylene glycol complex, aluminum sesquichlorohydrate/propylene glycol complex, aluminum chlorohydrate/polyethylene glycol complex, aluminum dichlorohydrate/polyethylene glycol complex, aluminum sesquichlorohydrate/polyethylene glycol complex, aluminum zirconium trichlorohydrate, aluminum zirconium tetrachlorohydrate, aluminum zirconium pentachlorohydrate, aluminum zirconium octachlorohydrate, aluminum zirconium trichlorohydrate/glycine complex, aluminum zirconium tetrachlorohydrate/glycine complex, aluminum zirconium octachlorohydrate/glycine complex, aluminum chloride, aluminum sulfate, and the like.

Usable examples of commercial products include, as aluminum zirconium chloridepropylene glycol complexes, “REZAL 36 PGP POWDER” and “REACH AZP-908 PG POWDER” (both, trade names, products of Reheis Inc.), “WESTCHLOR ZR41 PG ASP POWDER” (trade name, product of Westwood Chemical Corp.), and the like; and as aluminum zirconium chlorohydrateglycine complexes, “REACH AZP-908”, “REACH AZP-908SUF” and “REACH AZP-902” (all, trade names, products of Reheis Inc.), and the like.

Such antiperspirant agents can be used either singly or in combination. The antiperspirant agent may be contained preferably at from 10 to 70 wt %, more preferably at from 15 to 50 wt %, even more preferably at from 15 to 25 wt % based on the whole composition, because the effects of controlling perspiration and odors can be sufficiently obtained and the feel to the touch is also good.

It is to be noted that this “wt %” is determined through calculation by assuming the antiperspirant as an anhydrous metal salt, excluding water, glycine, glycine salt and any other complexing agent.

As the deodorant agent in ingredient (D), it is possible to use known substances and those capable of preventing or removing perspiration odors. Such deodorant agents can be antimicrobial agents (bactericides or fungicides), odor adsorbing materials, and the like.

Among deodorant actives, illustrative antimicrobial agents include alkyltrimethylammoniumbromide, cetylpyridinium chloride, benzethonium chloride, (diisobutylphenoxyethoxyethyl)dimethylbenzylammonium chloride, N-lauroylsarcosine sodium salt, N-palmitoylsarcosine sodium salt, N-myristoyl glycine, N-laurolylsarcossine potassium salt, trimethylammonium chloride, sodium aluminum chlorohydroxylactate, triethyl citrate, tricetylmethyl ammonium chloride, 2,4,4′-trichloro-2′-hydroxydiphenyl ether (triclosan), and 3,4,4′-trichlorocarbanilide (triclorocarban); diaminoalkylamides suchasL-lysine hexadecylamide; heavy metal salts, preferably zinc salts of citric acid, salicylic acid, piroctose and the like, and heavy metal salts of such acids and pyrithione, preferably zinc pyrithione, zinc phenolsulfate, farnesol, phenoxyethanol, isopropyl methylphenol, and the like.

Preferred antimicrobial agents include triclosan and isopropyl methyphenol. Such an antimicrobial agent may be contained preferably at from 0.01 to 10 wt %, more preferably at from 0.1 to 5 wt %, even more preferably at from 0.1 to 3 wt % based on the whole composition.

As the odor adsorbing materials, examples include carbonates and hydrogen carbonates such as alkali metal carbonates and alkali metal hydrogen carbonates, ammonium salts, tetraalkylammonium salts, and the like. The sodium salts and potassium salts of these odor adsorbing materials are more preferred. Such an odor adsorbing material may be contained preferably at from 0.001 to 60 wt %, more preferably at from 0.01 to 30 wt %, even more preferably at from 0.01 to 3 wt %. Insofar as the content of the odor adsorbing material falls within this range, the deodorizing performance of the stick composition can be improved without impairing its strength and feel to the touch.

In addition, organic or inorganic porous particles with a metal ion such as silver, copper, zinc or cerium carried thereon can also be used. They contain complexed globular particles of needles such as Cancrinite and their complex of heavy metal. They act not only as antimicrobial agents but also as odor adsorbing materials.

Further, the weight ratio, (B)/(D), of ingredient (B) to ingredient (D) may be preferably from 1/7 to 5/6, more preferably from 1/6 to 2/3, because such a weight ratio makes it possible to reduce a white residue further while retaining a good feel to the touch and a high level of antiperspirant deodorant ability.

The antiperspirant/deodorant stick composition according to the present invention may further contain a wax as ingredient (E). The inclusion of the wax brings about an improvement in high-temperature stability, and therefore, is preferred. Examples of such a wax include fatty acid esters, fatty acid amides, fatty acids, hydrogenated caster oil (castor wax), bees wax, montan wax, ozokerites, ceresin, paraffins, microcrystalline waxes, polyethylene wax, jojoba ester waxes, hydrogenated botanical oils, silicone waxes, and the like.

The silicone waxes include, but are not limited to, “AMS-C30” (trade name, C₃₀-C₄₅ malkyl dimethicone, product of Dow Corning Corporation), “SILCARE 41M80” (trade name, C₂₄-C₂₈ alkyl dimethicone, product of Clariant AG), “GESF1642” (trade name, C₃₀-C₄₅ alkyl dimethicone, product of General Electric Company), “KT-18” (trade name, dimethylstearylpolysiloxane, product of GE Toshiba Silicones, Ltd.), and the like.

As modified silicone waxes, it is also possible to use those disclosed in U.S. Pat. No. 5,654,362, U.S. Pat. No. 5,919,437, U.S. Pat. No. 5,976,514, U.S. Pat. No. 5,891,424, U.S. Pat. No. 6,060,546, U.S. Pat. No. 6,352,688, U.S. Pat. Application Publication No. 2003/0202949, and so on.

Among these, hydrogenated castor oil, fatty acids, silicone waxes and glycerol monostearate are preferred because they can impart adequate hardness and stability.

As the wax (E), such waxes can be used either singly or in combination. The wax (E) may be contained preferably at from 1 to 10 wt %, more preferably at from 2 to 8 wt % based on the whole composition because the composition thus obtained gains excellent stability and can provide a good feel to the touch.

The antiperspirant/deodorant stick composition according to the present invention may further contain a non-volatile cosmetic oil as ingredient (F). The inclusion of the non-volatile cosmetic oil is preferred in obtaining emollient effects. It is to be noted that the term “non-volatile” as used herein means a property not encompassed by the above-defined term “volatile”.

Examples of the non-volatile cosmetic oil include non-volatile silicones, hydrocarbon oils, fatty acids, high alcohols other than ingredient(A), ester oils, ether oils, mineral oils, polyethylene glycols, polypropylene glycols other than ingredient (B), and the like.

The non-volatile silicones include polyalkylsiloxanes, polyalkylarylsiloxanes, polyethersiloxane copolymers, and the like.

Examples of the non-volatile polyalkylsiloxanes include polydimethylsiloxanes having viscosities at 25° C. of from 5 to 100,000 mm²/s. Such non-volatile polydimethylsiloxanes are those represented by the following formula: 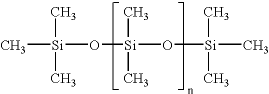
wherein n is such a number that a viscosity in a ambient is about 100,000 mm²/s or lower, preferably 500 mm²/s or lower, more preferably from 5 to 200 mm²/s, even more preferably from 5 to 50 mm²/s.

Illustrative usable non-volatile straight-chain silicones include “DOW CORNING 200” and “DOW CORNING 225” (both, trade names, products of Dow Corning Corporation), “SF-96” and “SF-18(350)” (both, trade names, products of GE Toshiba Silicones, Ltd.), “DM-FLUID” (trade name, product of Shin-Etsu Chemical Co., Ltd.), and the like.

Illustrative usable polyalkylarylsiloxanes include “SF-1550” (trade name, product of GE Toshiba Silicones, Ltd.), “556 COSMETIC GRADE FLUID” (trade names, products of Dow Corning Corporation), “KF56” (trade name, product of Shin-Etsu Chemical Co., Ltd.), and the like.

Illustrative non-volatile polyethersiloxane copolymers include polyoxyalkylene ether siloxane copolymers having a viscosity at 25° C. of from 1,200 to 1,500 mm²/s. A such copolymers, “SF-1188” and “SF-1288” (both, trade names, products of GE Toshiba Silicones, Ltd.), “KF6017” (trade name, product of Shin-Etsu Chemical Co., Ltd.), and the like can be used, for example.

Other non-volatile cosmetic oils include PPG-14butyl ether (a condensation product of propylene oxide (14 moles) and butyl alcohol (1 mole), for example, “FLUID AP” (trade name, product of Union Carbide Corporation) or “PROBTYL14” (trade name, product of Croda, Inc.); ester oils such as isopropyl myristate, isopropyl palmitate, cetyl octanoate, C₁₂-C₁₅ alkyl benzoate, isostearyl isostearate, octyldodecyl benzoate, isostearyl lactate, isostearyl palmitate and isobutyl stearate; octyl dodecanol; mineral oils; and the like.

The antiperspirant/deodorant stick composition according to the present invention may contain an unsaturated fatty acid having from 14 to 18 carbon atoms. The inclusion of such an unsaturated fatty acid makes it possible to adjust the hardness of the stick and to reduce the production of white residue, and therefore, is preferred. Examples of such an unsaturated fatty acid include myristoleic acid, palmitoleic acid, oleic acid, linoleic acid, linolenic acid, and the like, with oleic acid being preferred.

The unsaturated fatty acid may be contained preferably at from 0.01 to 1 wt %, more preferably at from 0.2 to 0.9 wt %.

As the non-volatile cosmetic oil (F), such non-volatile cosmetic oils can be used either singly or in combination. The non-volatile cosmetic oil (F) maybe contained preferably at from 1 to 30 wt %, more preferably at from 1 to 15 wt %, even more preferably from 1 to 7 wt % based on the whole composition, because sufficient emollient effects can be obtained and the feeling upon use is good.

The antiperspirant/deodorant stick composition according to the present invention can contain a humectant. No particular limitation is imposed on the humectant insofar as it is usable in conventional cosmetic preparations. Illustrative are glycerin, sorbitol, ethylene glycol, propylene glycol, and the like.

These humectants can be used either singly or in combination. The humectant may be contained preferably at from 0.1 to 2 wt %, more preferably at from 0.1 to 1 wt % based on the whole composition, because sufficient humectant effects can be obtained and the feeling upon use is good.

The antiperspirant/deodorant stick composition according to the present invention can additionally contain other ingredients usable in conventional antiperspirant/deodorant products, other personal care products or skin external preparations.

Such ingredients include, for example, dyes, colorants, emulsifiers, fragrances, preservatives, vitamins, coenzymes, antioxidants, coupling agents, solvents, surfactants, viscosity controlling agents, rinse adjuvants, application aids, pharmaceutically active agents, fillers, botanical extracts, and the like. For example, the ingredients disclosed in U.S. Pat. No. 4,049,792, U.S. Pat. No. 5,019,375, U.S. Pat. No. 5,429,816 or the like can be used.

The vitamins include vitamin Bs such as thiamine, riboflavin and pyridoxine. The coenzymes include thiamine pyrophosphate, flavin adenine dinucleotide, folic acid, pyridoxal phosphate, tetrahydrofolic acid, and the like. Vitamin A and its derivatives can also be used, and they include, for example, vitamin A palmitate, vitamin A acetate, other esters of vitamin A, β-carotene, and the like. Also usable are vitamin E and its derivatives, for example, vitamin E acetate, vitamin E nicotinate, other esters of vitamin E; vitamin D; and vitamin K.

The antioxidants include potassium sulfite, sodium hydrosulfite, sodium erythrobate, sodium hydrogenmetasulfite, sodium sulfite, propyl gallate, cysteine hydrochloride, butylated hydroxytoluene, butylated hydroxyanisole, and the like.

Examples of the fillers include talc, mica, sericite, kaolin, starch, hydrophobized starch such as “DRY FLO PC” (trade name, product of National Starch and Chemical Company), silicone resins such as “TOSPEARL” (trademark, product of GE Toshiba Silicones, Ltd.), fumed or colloidal silica such as “CAB-O-Sil M-5” (trade name, product of Cabot Corporation), non-fumed silica, and the like. These fillers can provide solid sticks with improved feeling upon use and a stabilized structure. The filler may be contained preferably at from 0.01 to 10 wt % based on the whole composition.

The antiperspirant/deodorant stick composition according to the present invention may contain, as a filler, clay and a clay-activating agent. Clay may be contained preferably at from 0.5 to 7 wt %, more preferably at from 1 to 5 wt % based on the whole composition.

Examples of the clay include montmorillonite clays, hydrophobically-treated montmorillonite clays, and the like. These montmorillonite clays contain montmorillonite mineral and have a suspended lattice. Such clays include bentonites, hectorites, colloidal magnesium aluminum silicates, and the like.

When a clay-activating agent is used, it may be contained preferably at from 0.2 to 1.5 wt %, more preferably at from 0.25 to 1.25 wt % based on the whole composition. The activating agent is generally a polar compound, and chemically activates clay. Such polar activating agents include propylene carbonate, ethanol and the like. Ethanol is preferred because it can enhance the property that the composition absorbs water. Clay and its activating agent may be added separately or as a mixture of the clay, the activating agent and a solvent. For example, “BENTONE GEL VSSV” (trade name) which is commercially available from Element is Specialties, Inc. is a mixture composed of about 77 wt % of cyclomethicone, 18 wt % of quarternium-18 hectorite and 5 wt % of SDA-40 alcohol (ethanol).

As a filler, porous inorganic particles may also be contained. Preferred porous inorganic particles include calcium silicate and silica, with those having pore volumes of 2 mL/g or greater being preferred. As such porous particles, “FLORITE R” (trademark, product of Tokuyama Corp.), “SUNPHERE H-33” (trade name, product of Asahi Glass Co., Ltd.) and the like can be mentioned. Such porous inorganic particles may be contained preferably at from 0.02 to 10 wt %, more preferably at from 0.05 to 5 wt %.

Examples of the fragrances include fragrance substances capable of masking perspiration odors and substances capable of providing the composition with desired fragrances. Such fragrances also include fragrances and fragrance chemicals including properfumes and deoperfumes suited for application to the skin. The content of a fragrance in the composition is an amount effective for imparting a desired fragrance or masking odors (the odor of the composition itself, and odors associated with human perspiration).

The antiperspirant/deodorant composition according to the present invention can also contain various organic and inorganic pigments. Examples of the organic pigments include various aromatic organic pigments such as azo, indigo, triphenylmethane, anthraquinone and xanthine dyes, which can be referred to as D&C and FD&C blue, brown, green, orange, red, yellow, violet, etc. The organic pigments also include insoluble metal salts of dyes, which are called “lakes”. Examples of the inorganic salts include ironoxides, ultramarine, chromium, chromium hydroxide, and the like.

The antiperspirant/deodorant composition according to the present invention can be produced in a desired form by a conventional method.

The antiperspirant/deodorant composition according to the present invention is an anhydrous composition. It is to be noted that the term “anhydrous” as used herein generally means to contain water less than about 10 wt %, preferably less than 8 wt %, more preferably less than 5 wt %, even more preferably less than 3 wt % in addition to water hydrated with a particulate solid.

The antiperspirant/deodorant composition according to the present invention is in the form of a solid stick. The term “solid” means that the hardness as measured by using a usual instrument is about 100 g or higher, generally about 200 g or higher. As a general method for the measurement of the hardness, the hardness can be measured at 25° C. and 40% relative humidity by using “FUDOH RHEO METER NRM-2010J-CW” (trade name, manufactured by Reotech Co., Ltd.). Its standard penetration plunger is made of stainless steel, has a diameter of 3 mm, and is commercially available as “Adapter No. 6” from Reotech Co., Ltd. The hardness indicates the greatest force required to move the standard plunger at 2 cm/min over a distance of 10 mm from a center of a surface of the antiperspirant/deodorant composition.

The antiperspirant/deodorant composition according to the present invention may be used by applying it to the axillae or other areas in an amount sufficient to control perspiration and/or odors. Onto each desired area on the skin, it may be applied preferably in an amount of from about 0.1 to 10 g, more preferably in an amount of from 0.1 to 5 g, even more preferably in an amount of from 0.1 to 1 g. Further, the antiperspirant/deodorant composition according to the present invention may be applied preferably once or twice a day, more preferably once a day to effectively control perspiration and/or odors.

The antiperspirant/deodorant composition according to the present invention may preferably have a clarity score of 3 or greater in a clarity test that uses artificial leather and employs talc as a standard.

Described specifically, talc (United States Pharmacopeia) was dispersed in decamethylcyclopentasiloxane to prepare slurries of the below-described concentrations. Respective talc slurries (0.1 g, each) were dropped through dropping pipettes onto artificial leather pieces within circles of 1.5 cm in diameter, followed by drying to provide standards. The slurry concentrations and scores were set in the following correlations.

| Slurry concentrations | Scores |
|---|---|
| 5.0 wt % | 1 |
| 2.5 wt % | 2 |
| 1.25 wt % | 3 |
| 0.625 wt % | 4 |

On the other hand, each antiperspirant deodorant stick composition was applied in an amount of 0.008±0.001 g on a similar artificial leather piece as those employed in the above-described preparation of the standards. After the application, the artificial leather piece with the antiperspirant deodorant stick composition applied thereon was exposed for 30 seconds to 40° C. and 75% humidity, and was then dried for 2 hours under environmental temperature and ambint (23 to 25° C., 25 to 60%). By visually observing the specimen and the above-described standards, the score of the test specimen was determined.

The following examples further describe and demonstrate embodiments of the present invention. The examples are given solely for the purpose of illustration and are not to be construed as limitations of the present invention.

### EXAMPLES

### Examples 1-10 & Comparative Examples 1-6

Anhydrous antiperspirant deodorant stick compositions of the formulations shown in Table 1 and Table 2 were produced, and were evaluated for a dry feel, non-stickiness, smoothness, and no production of white residue. The results are also shown in Table 1 and Table 2.

(Production Procedure)

Each anhydrous antiperspirant deodorant stick composition was produced as will be described hereinafter. The higher alcohols, wax, volatile cosmetic oil and non-volatile cosmetic oil(s) were mixed, and were then heated and stirred at a temperature high enough to fuse the higher alcohols and wax (about 80° C.) into a single liquid phase. The liquid phase was maintained at a temperature (about 65° C.) slightly higher than the solidification point of the system, and with stirring, the remaining ingredients other than ingredient (D) were added, followed by the addition of ingredient (D). Subsequent to thorough stirring, the mixture was poured into a container, and then allowed to solidify at room temperature.

Evaluation Method

(1) Dry feel, non-stickiness and smoothness

By three expert panelists, each stick was evenly applied in an appropriate amount onto their forearms, and its dry feel, non-stickiness and smoothness were organoleptically evaluated in accordance with the following standards. The results were each presented by the average of scores allotted by the three expert panelists.

(1-1) Dry feel (dry feel upon application)

4 Sufficient

3 Slightly insufficient

2 Insufficient

1 Very insufficient

(1-2) Non-stickiness (non-stickiness upon application and shortly after application)

5 Absolutely non-sticky

4 Non-sticky

3 Cannot be said either non-sticky or sticky

2 Slightly sticky

1 Sticky

(1-3) Smoothness (a smooth touch feel upon application)

5 Smooth

4 Slightly smooth

3 Cannot be said either smooth or not smooth

2 Not very smooth

1 Absolutely not smooth

(2) Non-production of white residue (visible whiteness after drying)

(2-1) Preparation of specimens

Each specimen was prepared as will be described hereinafter. On a piece of artificial leather (2×6 cm, “GOHI GT-202”, trade name, color number: 204; product of Daido Leather K.K.), the corresponding stick was stroked five times to apply it in an amount of 0.008±0.001 g. It is to be noted that the applied weight was determined from a difference between the weight of the stick before the application and that after the application. Subsequent to the application, the artificial leather piece was exposed for 30 seconds to 40° C. and 75% humidity and then dried for 2 hours under environmental temperature and environmental humidity (23 to 25° C., 25 to 60%).

(2-2) Preparation of standards

Talc (United States Pharmacopeia) was dispersed at predetermined concentrations in decamethylcyclopentasiloxane to prepare slurries. Respective talc slurries (0.1 g, each) were dropped through dropping pipettes onto similar artificial leather pieces within circles of 1.5 cm in diameter, followed by drying to provide standards. The slurry concentrations and scores were set in the following correlations.

| Slurry concentrations | Scores |
|---|---|
| 5.0 wt % | 1 |
| 2.5 wt % | 2 |
| 1.25 wt % | 3 |
| 0.625 wt % | 4 |

(2-3) Evaluation

By visually observing the specimens and the standards, the specimens were evaluated at intervals of 0.5 (7 grades)

TABLE 1

| | | Examples | | | | | | | | | |
|---|---|---|---|---|---|---|---|---|---|---|---|
| Ingredients (wt %) | | 1 | 2 | 3 | 4 | 5 | 6 | 7 | 8 | 9 | 10 |
| (1) | Stearyl alcohol | 22 | 22 | 22 | 22 | 22 | 15 | 17 | 25 | 27 | 30 |
| (2) | Behenyl alcohol | 0.5 | 0.5 | 0.5 | 0.5 | 0.5 | 0.5 | 0.5 | 0.5 | 0.5 | 0.5 |
| (3) | Hydrogenated castor oil | 4 | 4 | 4 | 4 | 4 | 4 | 4 | 4 | 4 | 4 |
| (4) | PPG (ave. m.w. 200)*¹ | | | | | | | | | | |
| (5) | PPG (ave. m.w. 400)*² | 7 | | | | | | | | | |
| (6) | PPG (ave. m.w. 1000)*³ | | 7 | | 2 | 16 | 7 | 7 | 7 | 7 | 7 |
| (7) | PPG (ave. m.w. 2000)*⁴ | | | 7 | | | | | | | |
| (8) | PPG (ave. m.w. 3000)*⁵ | | | | | | | | | | |
| (9) | PPG-14 butyl ether | 1 | 1 | 1 | 1 | 1 | 1 | 1 | 1 | 1 | 1 |
| (10) | Alkyl(C₁₂-C₁₅) benzoate | | | | | | | | | | |
| (11) | Decamethylcyclo- | 40.5 | 40.5 | 40.5 | 45.5 | 31.5 | 47.5 | 45.5 | 37.5 | 35.5 | 32.5 |
| | pentanesiloxane*⁶ | | | | | | | | | | |
| (12) | Aluminum zirconium | 25 | 25 | 25 | 25 | 25 | 25 | 25 | 25 | 25 | 25 |
| | tetrachlorohydrate. glycine | | | | | | | | | | |
| | complex*⁷ | | | | | | | | | | |

| | | | | | | | | | | |
|---|---|---|---|---|---|---|---|---|---|---|
| Total | 100 | 100 | 100 | 100 | 100 | 100 | 100 | 100 | 100 | 100 |
| Dry feel | 2.7 | 3.7 | 3.0 | 2.7 | 2.7 | 2.7 | 3.7 | 3.7 | 3.3 | 3.0 |
| Non-stickiness | 3.7 | 4.7 | 4.0 | 4.7 | 3.3 | 3.0 | 4.0 | 4.7 | 3.7 | 3.0 |
| Smoothness | 3.7 | 4.3 | 4.0 | 3.3 | 4.0 | 4.7 | 4.7 | 4.0 | 3.0 | 3.0 |
| Non-production of white | 3.5 | 3.5 | 3 | 3 | 4 | 3 | 3 | 4 | 4 | 4 |
| residue | | | | | | | | | | |

*¹“NEWPOL PP-200” (product of Sanyo Chemical Industries, Ltd.)
*²“NEWPOL PP-400” (product of Sanyo Chemical Industries, Ltd.)
*³“NEWPOL PP-1000” (product of Sanyo Chemical Industries, Ltd.)
*⁴“NEWPOL PP-2000” (product of Sanyo Chemical Industries, Ltd.)
*⁵“NEWPOL PP-3000” (product of Sanyo Chemical Industries, Ltd.)
*⁶“SH245” (product of Dow Corning Toray Co., Ltd.)
*⁷“REACH AZP-908SUF” (product of Reheis Inc.)

TABLE 2

| | Comparative Examples | | | | | |
|---|---|---|---|---|---|---|
| Ingredients (wt %) | 1 | 2 | 3 | 4 | 5 | 6 |
| (1) Stearyl alcohol | 22 | 22 | 12 | 35 | 17 | 17 |
| (2) Behenyl alcohol | 0.5 | 0.5 | 0.5 | 0.5 | 0.5 | 0.5 |
| (3) Hydrogenated castor oil | 4 | 4 | 4 | 4 | 4 | 4 |
| (4) PPG (ave. m.w. 200)*¹ | 7 | | | | | |
| (5) PPG (ave. m.w. 400)*² | | | | | | |
| (6) PPG (ave. m.w. 1000)*³ | | | 7 | 7 | | |
| (7) PPG (ave. m.w. 2000)*⁴ | | | | | | |
| (8) PPG (ave. m.w. 3000)*⁵ | | 7 | | | | |
| (9) PPG-14 butyl ether | 1 | 1 | 1 | 1 | 1 | 8 |
| (10) Alkyl(C₁₂-C₁₅) benzoate | | | | | 32.5 | |
| (11) Decamethylcyclo- | 40.5 | 40.5 | 50.5 | 27.5 | 20 | 45.5 |
| pentanesiloxane*⁶ | | | | | | |
| (12) Aluminum zirconium | 25 | 25 | 25 | 25 | 25 | 25 |
| tetrachlorohydrate. | | | | | | |
| glycine complex*⁷ | | | | | | |
| Total | 100 | 100 | 100 | 100 | 100 | 100 |
| Dry feel | 1.0 | 1.0 | 1.0 | 2.3 | 1.0 | 2.7 |
| Non-stickiness | 1.3 | 1.0 | 1.0 | 2.7 | 1.0 | 3 |
| Smoothness | 2.3 | 1.7 | 2.3 | 1.0 | 4.3 | 4.7 |
| Non-production of white | 3 | 2.5 | 2 | 4 | 2 | 2 |
| residue | | | | | | |

*¹“NEWPOL PP-200” (product of Sanyo Chemical Industries, Ltd.)
*²“NEWPOL PP-400” (product of Sanyo Chemical Industries, Ltd.)
*³“NEWPOL PP-1000” (product of Sanyo Chemical Industries, Ltd.)
*⁴“NEWPOL PP-2000” (product of Sanyo Chemical Industries, Ltd.)
*⁵“NEWPOL PP-3000” (product of Sanyo Chemical Industries, Ltd.)
*⁶“SH245” (product of Dow Corning Toray Co., Ltd.)
*⁷“REACH AZP-908SUF” (product of Reheis Inc.)

1. An anhydrous antiperspirant/deodorant stick composition comprising the following ingredients (A), (B), (C) and (D):
(A) from 15 to 30 wt % of a higher alcohol having from 12 to 26 carbon atoms,
(B) a polypropylene glycol having an average molecular weight of from 400 to 2,000,
(C) a volatile cosmetic oil, and
(D) an antiperspirant agent or deodorant agent.

2. The anhydrous antiperspirant/deodorant stick composition according to claim 1, comprising from3 to 15 wt % of said polypropylene glycol as ingredient (B).

3. The anhydrous antiperspirant/deodorant stick composition according to claim 1, wherein said volatile cosmetic oil is a volatile silicone.

4. The anhydrous antiperspirant/deodorant stick composition according to claim 1, wherein said antiperspirant agent as ingredient (D) is a complex of aluminum chlorohydrate or aluminum zirconium chlorohydrate and glycine.

5. The anhydrous antiperspirant/deodorant stick composition according to claim 1, wherein a weight ratio (B)/(D) of said ingredient (B) to said antiperspirant agent or deodorant agent as ingredient (D) is from 1/7 to 5/6.

6. The anhydrous antiperspirant/deodorant stick composition according to claim 1, further comprising: (E) from 1 to 10 wt % of a wax.

7. The anhydrous antiperspirant/deodorant stick composition according to claim 1, further comprising: (F) from 1 to 30 wt % of a non-volatile cosmetic oil.

8. The anhydrous antiperspirant/deodorant stick composition according to claim 1, wherein said ingredient (B) is a polypropylene glycol having an average molecular weight of from 400 to 1,000.

9. The anhydrous antiperspirant/deodorant stick composition according to claim 1, which has a clarity score of at least 3 in a clarity test that uses artificial leather and employs talc as a standard.
